(19) United States
(12) Patent Application Publication
(10) Pub. No.: US 2006/0240402 A1
(43) Pub. Date: Oct. 26, 2006

(54) Marine invertebrate gametes and embryos as an assay system for therapeutic screening

(76) Inventor: Robert E. Palazzo, Latham, NY (US)

Correspondence Address: ELMORE PATENT LAW GROUP, PC, 209 MAIN STREET, N. CHELMSFORD, MA, 01863, US

(21) Appl. No.: 11/369,174

(22) Filed: Mar. 6, 2006

Related U.S. Application Data
(60) Provisional application No. 60/660,080, filed on Mar. 9, 2005.

(51) Int. Cl. C12Q 1/00

(52) U.S. Cl. 435/4

(57) ABSTRACT

The invention provides methods of using of marine invertebrate gametes and preparations thereof for drug discovery and therapeutic screening.

### RELATED APPLICATION

This application claims the benefit of U.S. Provisional Application No. 60/660,080, filed on Mar. 9, 2005. The entire teachings of the above application are incorporated herein by reference.

### GOVERNMENT SUPPORT

The invention was supported, in whole or in part, by a grant R01 GM43264 from the National Institute of Health. The Government has certain rights in the invention.

### FIELD OF THE INVENTION

The invention relates to drug discovery and therapeutic screening assays for the identification of potential therapeutic agents, particularly those agents affecting the centrosome, centrosome-mediated or centrosome-related cellular processes.

### BACKGROUND OF THE INVENTION

Centrosomes and their counterparts (Spindle Pole Bodies, Nuclear Associated Bodies, etc.) are unique subcellular organelles involved in the organization of cytoarchitecture from yeast to man. They belong to a larger diverse group of organelles that share a common ability to nucleate and organize microtubules and are thus referred to as microtubule organizing centers or MTOCs. Features associated with MTOCs include organization of mitotic spindles, formation of primary cilia, progression through cytokinesis, and self-duplication once per cell cycle. Centrosomes bind many regulatory proteins, whose identities suggest roles in a multitude of cellular functions. Recently, MTOCs have been shown to be directly or indirectly involved in numerous fundamental cell processes including cell replication, cell migration, directed organelle traffic and maintenance of cell shape and polarity and are required for several regulatory functions including cell cycle transitions, cellular responses to stress, and organization of signal transduction pathways. While used as a model system for investigations of cellular processes, the centrosome has not been appreciated as a therapeutic target even though centrosome amplification occurs frequently in almost all types of cancer, and is considered as the major contributing factor for chromosome instability in cancer cells. Consequently, there remains a long felt need for assays and screening methods that allow for the identification of therapeutic agents which target the centrosome.

The methods of the present invention not only solve this problem but, in the combinations disclosed herein, allow for the identification of the particular stage or transition of development at which a potential therapeutic agent or drug is acting to block or alter centrosome-mediated or centrosome-related cellular processes.

The combinatorial use of the methods and screening assays of the present invention result in a highly efficient and inexpensive drug discovery platform.

### SUMMARY OF THE INVENTION

The invention provides methods of using marine invertebrate gametes and preparations thereof in assays for drug discovery and therapeutic screening.

### DETAILED DESCRIPTION OF THE INVENTION

For the purposes of promoting an understanding of the principles of the invention, reference will now be made to particular embodiments and specific language will be used to describe the same. It will nevertheless be understood that no limitation of the scope of the invention is thereby intended. It should also be understood that the embodiments described herein are not mutually exclusive and that features from the various embodiments may be combined in whole or in part in accordance with the invention.

The present invention provides for the use of marine invertebrate eggs, embryos and sperm, and preparations thereof, particularly Spisula solidissima and/or Mulinia lateralis, sea urchins, mussels, other clams, starfish, other marine invertebrates for drug discovery and therapeutic screening. Surprisingly, assay methods and techniques for studying the centrosome in Spisula solidissima (the Atlantic surf clam) have been found to be unexpectedly useful in the identification of potential therapeutic agents, their targets and mechanisms of action. In one embodiment, the therapeutic screening comprises rapid or high throughput screening that may be automated. High-throughput screening allows a large number of potential therapeutic agents to be tested. For example, a large number of potential therapeutic agents can be tested individually using rapid automated techniques or in combination using a combinational library. Individual compounds demonstrating an effect in the assays of the present invention can be obtained by purifying and retesting fractions of the combinational library. Thus, thousands to millions of agents can be screened in a single day. Methods of automation are known to those skilled in the art

At the cellular level, the processes involved in the development of marine invertebrate eggs into an embryo are analogous to the processes that occur in mammalian cells. For this reason the information gained from the methods of screening disclosed herein can be used to accelerate the processes involved in drug discovery as they are readily transferable to mammalian systems. It is also contemplated that other eukaryotic systems may be employed in the same manner as the marine system described herein. The present invention therefore contemplates the use of the MIGS (marine invertebrate gamete system) as a component of a drug discovery or screening platform for the identification of therapeutic agents. The combination of the MIGS with known screening methods and drug discovery platforms for the identification of anti-mitotic agents is particularly preferred.

The present invention also has the advantage of simplified real-time readout as the cellular processes being evaluated are well characterized and can be visualized using various types of light microscopy. In one embodiment of the invention, visualization of cellular processes under investigation can include any of several types of microscopy including but not limited to bright field, polarized light, phase-contrast, fluorescence, Nomarski, and differential-interference-contrast (DIC). Utilization of these types of microscopy to cellular visualization is known in the art.

In one embodiment of the invention, light microscopy is used to assay in vitro and in vivo effects of the potential therapeutic agent or drug on a myriad of centrosome-mediated and centrosome-related cellular processes including nuclear envelope (germinal vesicle) breakdown, centrosome assembly and function, spindle assembly, cell and embryo cleavage.

The similarity of Spisula and Mulinia as organisms, including appearance, oocyte stage of arrest, mechanisms of oocyte activation including parthenogenic activation using KCl, the calcium requirement for activation, and the timing of development after fertilization suggest that Spisula and Mulinia may be distantly related in terms of evolution. The only difference between these organisms appears to be the size of the adult organism and the time required before organisms can become reproductive, i.e. generate gametes egg and sperm, which occurs within 60 days or less for Mulinia but requires longer time for Spisula. The organisms are so closely related that sperm and egg of each organism can be used in cross fertilizations to yield hybrid embryos. In one embodiment of the invention, Spisula and Mulinia gametes are cross fertilized to produce hybrid organisms useful in the assays and methods of the invention. These organisms may be produced from the sperm of Spisula and the eggs of Mulinia or from the eggs of Spisula and the sperm of Mulinia. These hybrid organisms are useful as research tools and in other commercial applications.

The marine invertebrate gamete system is advantageous over current systems of screening with cultured mammalian cells for several reasons. First, in contrast to mammalian cells, marine invertebrate eggs are naturally synchronous. Spisula solidissima eggs, for example, are arrested at the G2/M transition stage of the cell cycle. The present invention therefore contemplates the use of the marine invertebrate gamete system in research and screening of potential therapeutic agents which are known to affect or are suspected to alter cell-cycle progression.

Second, the clarity of the marine invertebrate eggs enables cytological analysis with light microscopy thereby increasing the speed and ease of analysis. In one embodiment, the invention disclosed herein includes the use of light microscopy for visualization but also contemplates the use of other indirect methods of measuring phenotypic endpoints including, but not limited to Fluorescence Activated Cell Sorting (FACS), spectroscopy, luminometric, photo-optical, and other approaches allowing for visualization.

Third, large quantities of marine eggs cells can be obtained at significantly lower cost than comparable amounts from mammalian systems. Finally, the speed and ease of preparation of components of the system make the marine invertebrate gamete system extremely attractive to rapid or high-throughput screening applications. Consequently, the marine invertebrate gamete system disclosed herein provides more material with less labor and less cost and offers a more robust readout thereby meeting the long felt need for methods of identifying potential therapeutic agents or drug compounds for use in the treatment of centrosome-mediated or centrosome-related disorders. For the foregoing reasons, the present invention is used in basic research and in the preparation of commercially available kits and assays for applications in high-throughput systems.

Identification of the Developmental Stage or Transition of Drug Block.

Since Spisula is commercially harvested, liters of preparation of gametes, embryos, and derived cell extracts and/or milligram quantities of functional centrosomes can be prepared from specific stages in the meiotic cell cycle as is known in the art that can be stored in freezers with no significant loss in relevant biological activity, making for a convenient experimental system. These lysates contain uniform maternal centrosomes at one specific stage in their maturation process, and are capable of inducing paternal centrosome maturation in vitro.

Therefore, the marine invertebrate gamete system disclosed herein has the advantage of being able to distinguish the stage or transition at which a potential therapeutic agent is acting in the cellular progression from egg to embryo. Briefly these stages include germinal vesicle breakdown, meiotic spindle assembly, polar body formation, mitotic spindle assembly for fertilized embryos) and cleavage events. In one embodiment of the invention, Spisula oocytes are treated with one or more potential therapeutic compounds and over a timecourse are visually monitored for effects on germinal vesicle breakdown, spindle assembly, polar body formation, aster formation, or cleavage events using light microscopy. In one embodiment the treatment of synchronized oocytes occurs before germinal vesicle breakdown. In one embodiment treatment occurs after germinal vesicle breakdown but before spindle assembly. In one embodiment, treatment occurs after spindle assembly but before polar body formation. In one embodiment treatment occurs after polar body formation but before embryonic cleavage. In one embodiment, treatment occurs during any of the several cleavage events in the embryo. In one embodiment, this identification is part of a unified screening system that can begin with the effect on the whole cell and which can be refined to determine the effect on biochemically isolated subcellular components. For example, the inhibition of spindle formation may result from a drug that affects tubulin polymerization or from a drug that affects the centrosome thus preventing microtubule nucleation. Although several drugs are known that inhibit tubulin polymerization, drugs that specifically target the centrosome have not yet been reported. The marine invertebrate gamete system described herein enables the identification of drugs targeting any of the stages or transitions in the development from egg to embryo as well as those targeting the centrosome. Being able to identify drugs with alternate targets is important for identifying drugs that can be used, for example, when a tumor develops resistance to a particular drug known to target a certain stage or transition.

Potential Therapeutic Agents of the Invention.

The in vitro assays and combinations thereof described herein enable screening of a variety of potential therapeutic agents. As used herein the term “potential therapeutic agent or therapeutic agent” is defined as any compound or library of compounds that are screened or tested in the marine invertebrate gamete system and includes compounds which are known therapeutics, and metabolites thereof. In one embodiment, libraries of potential therapeutic agents are screened. Types of molecules that could be identified are those that target the critical protein required for microtubule nucleation in all eukaryotic cells, gamma-tubulin, or unknown proteins required for centrosome assembly. Alternatively, molecules that lead to disruption of centrosome structure or assembly by inhibiting enzyme regulators present in cells and cell extracts may also be identified. New compounds that inhibit alpha/beta-tubulin (microtubule) polymerization, already known to be effective therapeutic agents, for example, anti-cancer agents, can also be identified by this method. These libraries will be screened for their ability to disrupt various aspects of spindle assembly either in whole cells, complex cell-free extracts, and in defined media. It is also contemplated by the present invention that once identified, lead compounds will be synthesized in bulk for use in basic cell biological research, as affinity agents for biochemical approaches to identify novel target molecules, and ultimately for the development of drug candidates for Phase I clinical trials.

Potential therapeutic agents include, but are not limited to, the classes of compounds comprising synthetic inorganic and organic compounds, proteins, peptides, polypeptides, DNA and RNA nucleic acid sequences having therapeutic, prophylactic or diagnostic activities and antibodies.

The terms “polypeptide,” “peptide,” and “protein” are used interchangeably herein to designate a linear series of amino acid residues connected one to the other by peptide bonds between the alpha-amino and carboxy groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids, which include, but are not limited to those listed in 37 CFR .sctn.1.822(b)(4). Peptides to be screened by the methods of the present invention comprise from 1-50 amino acids, whereas proteins or polypeptides comprise 50 amino acids or more.

As used herein “nucleic acid sequences” include genes, antisense molecules which bind to complementary DNA or RNA and inhibit transcription, siRNA (both single and double stranded), aptamers and ribozymes.

The term “DNA and RNA nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences, as well as the sequence explicitly indicated. Nucleic acid sequences to be screened by the methods disclosed herein comprise from about 2 to about 50 deoxyribonucleotides or ribonucleotides.

The term “antibody” is meant to include polyclonal antibodies, monoclonal antibodies (mAbs), chimeric antibodies, anti-idiotypic (anti-Id) antibodies to antibodies that can be labeled in soluble or bound form, as well as fragments, regions or derivatives thereof, provided by any known technique, such as, but not limited to, enzymatic cleavage, peptide synthesis or recombinant techniques.

It is also understood that any of these potential therapeutic agents may be chemically modified to improve any one of several properties. Methods of chemical modifications of nucleic acids are known in the art and include modifications to the subunits of the compounds such as the base or sugar for nucleic acids or the amino acid units of peptides and proteins. Modifications also include modifications such as the addition of conjugate moieties.

The terms “isolated” and “purified” refer to material which is substantially or essentially free from components which normally accompany it as found in its native state.

Applications of the potential therapeutic agents identified in the assays and methods of the present invention include but are not limited to 1) anti-cancer agents (anti-mitotic drugs, anti-centrosome drugs, and the like) comparable to Taxol and others as yet unknown, 2) 5HT blockers (serotonin receptor agonists or antagonists) that could be useful for the treatment of neuronal based and other diseases or as neuromodulators, 3) calcium channel blockers for the use in numerous disorders including hypertension, heart disease and others and 4) compounds that act as anti-fertility agents or those that prevent embryonic development, including possible male and female contraceptives, or to reverse infertility or as compounds that block embryonic development after fertilization has already occurred.

The potential therapeutic agents to be screened or tested can have a variety of biological activities, such as vasoactive agents, neuroactive agents, hormones, anticoagulants, immunomodulating agents, cytotoxic agents, chemotherapeutic, anti-tumor, anti-metastatic, prophylactic agents.

Abbreviations:

The following abbreviations are used herein: GVBD, germinal vesicle break down; MIGS, marine invertebrate gamete system; TURC, tubulin ring complex; ASW, artificial sea water; MNP, microtubule nucleation potential.

The MIGS (Marine Invertebrate Gamete System) Drug Discovery Platform

In one embodiment of the present invention a screening method for the identification of potential therapeutic agents is disclosed. This screening method involves the novel combination of assays and methods based on the use of gametes and embryos (in particular oocytes and preparations thereof) from invertebrates, preferably a commercially harvested marine clam, Spisula solidissima. These assays are described in and follow the teachings of Palazzo, R. E., Vaisberg, E. A., Weiss, D. G., Kuznetsov, S. A. and Steffen, W. (1999) J. Cell Sci. 112: 1291-1302; Palazzo, R. E., Vaisberg, E., Cole, R. W. and Rieder, C. L. (1992); Science. 256: 219-221; Vogel, J. M., Stearns, T., Rieder, C. L. and Palazzo, R. E. (1997) J. Cell Biol. 137: 193-202; Schnackenberg, B. J., Khodjakov, A., Rieder, C. L. and Palazzo, R. E. (1998) Proc. Natl. Acad. Sci. USA. 95: 9295-9300; Wu, X. and Palazzo, R. E. (1999) Proc. Natl. Acad. Sci. USA. 96: 1397-1402; and Schnackenberg, B. J. and Palazzo, R. E. (1999) Biology of the Cell. 91: 429-438, each of which are incorporated by reference in their entirety.

In one embodiment, the marine claim, Mulinia lateralus, can be used alone or in combination with the Spisula solidissima clam. In the combination it is contemplated that cross fertilization of Mulinia and Spisula gametes offer the advantage of hybrid organisms, further expanding the applications of the system described herein.

In one embodiment of the invention, the whole gamete (oocyte or sperm) and embryo assays described herein are used to identify potential therapeutic agents targeting the molecular events that lead to oocyte activation (in the case of serotonin, calcium channel blockers, etc.) spindle assembly and function and cell cleavage. As such, whole egg assays could be used as a first step in the screening a potential therapeutic compound or drug. The availability of a large population of synchronous cells is a major advantage of the marine invertebrate egg system. These cells can be easily activated into meiosis and/or mitosis as needed.

In another embodiment of the invention, cell-free functional reconstitution assays are used either in series with or parallel to the whole cell, gamete, or embryo assays to identify agents targeting nuclear envelope breakdown and chromosome release, centrosome maturation, centrosome-dependent microtubule nucleation and aster formation, disassembly and reassembly of centrosome microtubule nucleation potential and/or chromosome capture by centrosome microtubules, chromosome separation during meiosis or mitosis, and cleavage.

In one embodiment of the invention, the combination of whole cell (oocyte or sperm), embryo, and cell-free functional reconstitution assays are employed to identify the particular stage or transition of development being targeted by the therapeutic agents being tested.

In one embodiment the assays are employed in a series that comprises 1) whole oocyte and embryo (live cells and embryo assays) to visualize nuclear envelope breakdown, aster formation, spindle assembly and cleavage, 2) nuclear breakdown, aster formation, and spindle assembly in lysates prepared from these cells (it is understood that no cleavage can occur outside live cells), 3) defined assays using isolated centrosomes reconstituted with centrosome-free extracts prepared from any cell type, 4) defined assays using centrosome fragments and centrosome-free extracts prepared from any cell type (centrosome assembly assays), 5) fertilization assays using sperm and egg, and sperm centrosome assembly assays both in whole eggs and embryos, and 6) in vitro using denuded sperm heads and lysates as in 2, 3, and 4 above, 7) embryonic development overall.

The simplicity of these in vitro assays, the speed of reactions (complete spindle assembly within 10-15 min), and the large quantities of biological material available (liters of lysate and billions of centrosomes and chromosomes yearly) make the Spisula system ideal for screening to identify novel therapeutic agents, preferably anti-mitotic agents and unknown molecular targets in cells.

In another embodiment of the invention, certain aspects of spindle assembly, particularly those relating to centrosome assembly and chromosome capture by microtubules, are reconstituted in hybrid systems using centrosomes isolated from Spisula oocytes and molecules and chromosomes isolated from mammalian transformed or non-transformed cells, including human cells. These hybrid mammalian system assays can be performed in conjunction with or parallel to the whole invertebrate cell and embryo assays and the cell-free reconstitution assays allowing the identification of potential therapeutic agents, preferably anti-mitotic agents that specifically target mammalian proteins required for cell division. In another embodiment of the invention, parallel assays for disruption of microtubule polymerization using purified alpha/beta-tubulin subunits (a tubulin polymerization assay) can be used to screen out microtubule poisons, allowing the selection of novel centrosome-specific or anti-centrosome drugs.

According to the invention, once an agent has been identified as a potential therapeutic agent, the compound can be further tested for its effects on mammalian cells in vitro or in vivo. For example, compounds identified as inhibitors of centrosome-dependent microtubule nucleation, centrosome assembly, spindle assembly, and/or cleavage using the Spisula based screening method can then be tested for their effects on human cultured tumor cells, such as HeLa cells. For example, HeLa cells may be chilled to 4° C. to depolymerize microtubules, incubated with the identified potential therapeutic compound(s), and warmed to 37° C. In control cells, microtubules will rapidly assemble from centrosomes upon warming, and this would be blocked in the presence of a centrosome inhibitor. It is also contemplated that in vivo studies can be performed using any of the known model sytems for the disease, disorder or condition of interest.

Once identified, promising compounds can then be sent for resynthesis, preliminary optimization using a focused library to determine structure-activity relationships, derivatized for cell delivery if necessary, and/or retested for relevant physiologic and/or phenotypic outcomes (i.e. cytotoxicity, pharmacokinetic and pharmacodynamic properties, and the like). Collectively, the assays disclosed herein may be run in combination (either in series or in parallel) as a drug discovery or high throughput screening platform for the identification of novel drugs or for the identification of the mechanism of action of known drugs.

Whole Egg Assay and Lysates Preparation

Oocytes are prepared as generally described in Allen R D. (1953) Biol Bull. 105:213-239; Palazzo, R. E., Vaisberg, E., Cole, R. W. and Rieder, C. L. (1992) Science. 256: 219-221; Vogel, J. M., Stearns, T., Rieder, C. L. and Palazzo, R. E. (1997) J. Cell Biol. 137: 193-202; and Schnackenberg, B. J. and Palazzo, R. E. (1999) Biology of the Cell. 91: 429-438.

Briefly, adult Spisula or Mulinia are collected from the Atlantic Ocean, often by the Marine Resources Department of the Marine Biological Laboratory (Woods Hole, Mass.) or from commercial fisherman and maintained in flow-through sea water tanks at ˜13° C. containing artificial sea water (ASW). Oocytes are then dissected from ripe ovaries, passed through cheese cloth, and washed in sea water by three cycles of suspension/sedimentation. Oocytes are then activated by treatment with KCl for 4 min, and lysates are prepared as previously described by Palazzo et al. (Palazzo, R. E., J. B. Brawley, and L. I. Rebhun (1988). Zool. Sci. (Tokyo). 5: 603-611).

Aster formation in oocytes, embryos, and lysates is assessed with polarized light microscopy by adding 3% hexylene glycol to small aliquots and warming to 24° C. (Palazzo, R. E., J. B. Brawley, and L. I. Rebhun (1988). Zool. Sci. (Tokyo). 5: 603-611). The remaining lysate is aliquoted, snap frozen, and stored at −80° C. Frozen lysates retain the ability to assemble asters after years of storage.

Oocyte lysates are prepared from non-activated oocytes as generally described in Palazzo, R. E., Vaisberg, E., Cole, R. W. and Rieder, C. L. (1992) Science. 256: 219-221; Wu, X. and Palazzo, R. E. (1999) Proc. Natl. Acad. Sci. USA. 96: 1397-1402) or from oocytes that are parthenogenetically activated with KCl for 4 minutes as described in Palazzo, R. E., Vaisberg, E., Cole, R. W. and Rieder, C. L. (1992) Science. 256: 219-221; Vogel, J. M., Steams, T., Rieder, C. L. and Palazzo, R. E. (1997) J. Cell Biol. 137: 193-202; and Palazzo, R. E. and Vogel, J. M. (1999) In Mitosis and Meiosis: Methods in Cell Biology, vol. 61. C. L. Rieder, editor. Academic Press, Inc. 35-56.

In one embodiment of the invention, oocyte activation to induce GVBD, aster formation, spindle assembly, and cleavage can be induced by 5-hydroxy triptamine (5-HT, serotonin), fertilization, or depolarization of the cell plasma membrane by treatment with KCl when cells are incubated in sea water. This requires the entry of calcium into the cell after treatment with these agents. Thus, the GVBD, or events subsequent to GVBD, can be used as indicators for hormone-receptor interactions (serotonin), effect of calcium channel blockers (resulting in a block of GVBD and subsequent events after treatment with KCL, 5HT or sperm), or agents that block fertilization generally. Therefore the present invention can be used not only to screen for antimitotic agents but also putative neuromodulators (serotonin inhibitors and the like), cardiomodulators, vasoactive agents (calcium channel blockers) and anti-fertility agents.

Centrosome-Free Extracts

Briefly, to make centrosome-free extracts, frozen-stored lysates are thawed on ice and centrifuged three times at 39,000×g at 4° C. to clarify. Supernatants are collected and this process repeated three times.

High speed, centrosome-free extracts are prepared by diluting lysates 1:1 in PEM buffer or reassembly buffer (RAB; 100 mM Pipes, 1 mM EGTA, 5 mM MgSO4, pH 6.9) and centrifuging at 100,000×g for 1 hour at 4° C. in a TLA-100.3 rotor (Beckman Instruments, Palo Alto, Calif., USA) as described in Schnackenberg, B. J., Hull, D. R., Balczon, R. D. and Palazzo, R. E. (2000) J. Cell Sci. 113: 943-953. The supernatants are then collected and either used immediately or stored at −80° C. until further use. The final supernatant/extract collected and used in the assays for recovery of microtubule nucleation potential (MNP). When needed, these extracts are diluted 1/20 in PEM containing 20 μM colchicine. In some experiments, samples are supplemented with 5-50 mM 6-dimethylaminopurine (6-DMAP). For experiments involving EDTA, the 100,000×g extract is diluted in PE buffer (5 mM Pipes/1 mM EGTA, pH 7.2) containing 20 μM colchicine and 5-100 mM EDTA.

It is known that fertilization of oocytes with sperm or parthenogenic activation with KCl leads to nuclear envelope (germinal vesicle) breakdown (GVBD) then to first meiotic spindle assembly then to spindle migration then to polar body formation with 1st polar body then 2nd polar body being formed. If the oocyte is fertilized with sperm, then mitotic spindles are formed. This leads to series of cleavages that will ultimately result in the formation of an embryo. The present invention contemplates a screening method which results in the identification of the particular stage in the egg-to-embryo development pathway at which a potential therapeutic agent acts.

Centrosome Isolation/Preparation

Centrosomes are isolated and prepared according to the methods of Vogel et al. (Vogel, J. M., Stearns, T., Rieder, C. L. and Palazzo, R. E. (1997) J. Cell Biol. 137: 193-202). Briefly, centrosomes are isolated from lysates by sucrose density-gradient centrifugation using a modification of the procedures known in the art for isolation of mammalian centrosomes. As such, lysates are thawed on ice, and aliquots are removed and tested for aster formation using hexylene glycol and polarized light microscopy as previously described. For each preparative gradient, 2-3 ml of crude lysate (˜9-18×10⁶ centrosomes) are diluted with 0.60 vol of aster buffer (Aster Buffer: 20 mM sodium-Pipes, pH 7.2, 100 mM NaCl, 5.0 mM MgSO₄), resuspended, and centrifuged (5,500 g/4° C./10 min) using a rotor (model JS 13.1; Beckman Instruments). Supernatants are collected, diluted to 50.2% sucrose by addition of a stock solution of 70% (wt/wt) sucrose in PEM (5 mM potassium-Pipes, pH 7.2, 1 mM EGTA, 1 mM MgSO₄) and loaded onto a two-step gradient consisting of 66% (3.0 ml) and 52.5% (5.0 ml) sucrose steps. Gradients are centrifuged at 70,000 g at 4° C. for 90 min using a rotor (model SW-28; Beckman Instruments). Fractions (1.0 ml) are collected at 4° C. by bottom puncture of centrifuge tubes, and sucrose density is determined with the use of a hand-held refractometer.

Fractions are tested for centrosome content based on their ability to organize microtubules in the form of radial astral arrays when reconstituted with tubulin media. Thus, 10 μl aliquots from each fraction are diluted into 40 μl of tubulin media (0.3-0.7 mg/ml three-cycled sea urchin tubulin in RAB buffer containing 2 mM GTP) and incubated for 10 min at ambient temperature, and asters are counted using a hemacytometer viewed with a polarized light microscope. The fraction containing the highest density of centrosomes (centrosome fraction) is used directly, or snap frozen in liquid nitrogen and stored at −80° C. for further use.

Tubulin Preparation

Sea urchin (Strongylocentrotus purpuratus) microtubules are prepared by three cycles of polymerization/depolymerization as previously described (Suprenant, K. A., and J. C. Marsh (1987) J. Cell Sci. 87: 71-84). Aliquots of ˜0.5-2.0 mg/ml protein are stored in 100 mM potassium-Pipes, pH 6.9, 1 mM EGTA, 5 mM MgSO₄, 2 mM GTP (reassembly buffer) at −80° C. Three-cycled tubulin is diluted with reassembly buffer to 0.7 mg/ml for all functional assays unless otherwise stated.

Three-cycled tubulin can also be prepared from Spisula oocyte lysates using a modification of the procedure described in Suprenant, K. A. (1989) Exp. Cell Res. 184: 167-180. Lysates are thawed and diluted with 0.8 vol of dilution buffer (100 mM potassium-Pipes, pH 7.2, 4 mM EGTA, 1 mM MgSO₄, 1 mM DTT) containing 1 mM GTP, 10 mg/ml leupeptin, and 0.2 mM phenylmethylsulfonyl fluoride. Diluted lysate is then gently resuspended on ice and centrifuged (39,000 g/2° C./30 min) using a rotor (model JA20; Beckman Instruments Fullerton, Calif.) to clarify. Supernatants are collected and three-cycled tubulin prepared as previously described (Suprenant, K. A. (1989) Exp. Cell Res. 184: 167-180).

When necessary, sea urchin and/or Spisula tubulin is purified from three-cycled tubulin by polymerizing in the presence of Na-glutamate (Simon, J. R., S. F. Parsons, and T. Salmon (1992) Cell Motil. Cytoskel. 21: 1-14), followed by successive cycles of polymerization/depolymerization. The final five-cycled microtubules are pelleted through a cushion of 30% glycerol/reassembly buffer. Pellets are aspirated dry, snap frozen in liquid nitrogen, and stored at −80° C.

Microscopy and Visualization

The sequence of events from germinal vesicle breakdown to spindle assembly to polar body formation through fertilization and embryonic divisions as outlined above can be visualized via numerous forms of light microscopy. These forms of light microscopy are known in the art. The result of adding of agents which interfere with the normal progression of events can result in change in the timing of the progression between stages or arrest/block of progression to the next stage in the cell cycle and these alterations can also be visualized using light microscopy.

Electron microscopy tomography of centrosomes is described in Vogel, J. M., Steams, T., Rieder, C. L. and Palazzo, R. E. (1997) J. Cell Biol. 137: 193-202. Briefly, centrosome fractions are diluted into 2 vol of 3% glutaraldehyde in PEM buffer and incubated on ice for 10 min. After fixation, centrosomes are concentrated by centrifugation, postfixed with 1% OsO₄, dehydrated, and embedded as described previously for Spisula asters. (Palazzo, R. E., J. B. Brawley, and L. I. Rebhun (1988) Zool. Sci. (Tokyo). 5: 603-611). Semi-thick (200-300 nm) serial sections are then cut from each preparation. Each section is subsequently mounted in the center of a formvar-coated slot grid on which 15-nm colloidal gold had been lightly deposited to facilitate subsequent micrograph alignment. Sections are next stained with uranyl acetate and lead citrate. Selected regions within a section, containing the area to be reconstructed, are irradiated at 400 kV and normal beam intensity in an intermediate voltage electron microscope (IVEM) (model JEM-4000 FX; JEOL U.S.A., Inc., Peabody, Mass.) until the initial mass loss was completed (˜10 min). They are then photographed on SO 163 film, using a tilt-rotation stage, around two orthogonal (x and y) axes at increments of 2° over a ±60° range. To limit specimen deformation during sequential photography, the specimen is further irradiated only during image recording. 122 tilt images can be used for each reconstruction, with 61 images around the x-axis and 61 images around the y-axis. Images are then scanned so that each pixel was 2.6 nm and each image was 800×800 pixels. Three-dimensional tomographic reconstructions are then calculated by methods previously described in detail (Penczek, P., M. Marko, K. Buttle, and J. Frank (1995) Ultramicroscopy. 60: 393-410 and McEwen, B. F., J. Arena, J. Frank, and C. L. Rieder (1993) J. Cell Biol. 120: 301-312). Images are displayed in negative form to enhance the contrast.

Immunofluorescence

Immunofluorescence studies are performed according to the teachings of Wu, X. and Palazzo, R. E. (1999) Proc. Natl. Acad. Sci. USA. 96: 1397-1402 and Vogel, J. M., Steams, T., Rieder, C. L. and Palazzo, R. E. (1997) J. Cell Biol. 137: 193-202).

Briefly, centrosome proteins are localized by immunofluorescence as previously described (Mitchison, T. J., and M. Kirschner. (1986) Methods Enzymol. 134: 261-268; Palazzo, R. E., E. Vaisberg, R. W. Cole, and C. L. Rieder. (1992) Science (Wash. D.C.). 256: 219-221). The following modifications can be made. Aliquots of the centrosome fraction are reconstituted with tubulin media and incubated at room temperature. Samples are then fixed for 5 min at room temperature by adding 1% glutaraldehyde (Ted Pella, Inc., Redding, Calif.) in PBS (10 mM Na₂HPO₄, 1.8 mM KH₂PO₄, 136 mM NaCl, 2.6 mM KCl, pH 7.2). Fixed samples are then layered onto a 30% glycerol/PBS cushion, and centrosomes are centrifuged onto polylysine-coated coverslips at 8,000 g using a rotor (model JS 13.1; Beckman Instruments) according to the teachings of Mitchison and Kirschner (Mitchison, T. J., and M. Kirschner (1984) Nature (Lond.). 312: 232-237). The coverslips are incubated in ice-cold methanol for 5 minutes and then washed three times with PBS to rehydrate. All subsequent steps use PBS as buffer. Coverslips are then blocked with 5% BSA for 30 min, followed by application of the primary antibody. Various tubulin proteins are detected and localized using commercially available antibodies. Detection of antigen/antibody complexes is accomplished using either anti-mouse FITC or anti-rabbit rhodamine conjugates (Calbiochem, La Jolla, Calif.). Images are acquired with a standard fluorescence microscope or a laser scanning confocal microscope.

Sperm Aster Assay

Testes are removed from male clams and kept cool on ice. Released sperm are collected and stored at 4° C., and used for fertilization within 5 hours. The sperm aster assay is performed according to the methods disclosed by Wu, X. and Palazzo, R. E. (1999) Proc. Natl. Acad. Sci. USA. 96: 1397-1402. Warming of sperm with centrosome-free lysate results in the formation of asters which can be visualized via microscopy.

Fertilization and Fixation of Sperm and Embryos.

According to the teachings of Wu and Palazzo (Wu, X. and Palazzo, R. E. (1999) Proc. Natl. Acad. Sci. USA. 96: 1397-1402), oocytes are suspended in ASW containing 10 mM of Tris base (pH 8.0). Sperm are diluted in ASW with the Tris base, test fertilizations are conducted, and final fertilization and development are allowed to proceed at room temperature according to the method of Allen. Embryos can be followed through normal development in real time using standard light or fluorescence microscopy. For more detailed localization of specific molecules of interest, at different time points after fertilization, 2-3 drops of the sperm/embryo/sea-water suspension are loaded onto poly-L-lysine coated, round, 12-mm coverslips (BellCo Glass, Vineland N.J.). The sperm and embryos are allowed to attach for 4 minutes, and the coverslips are transferred into drops of 0.5 ml of extraction buffer (100 mM Pipes, pH 6.9/1 mM MgSO₄/5 mM EGTA/1 mM DTT/10% glyceroly 0.1% Nonidet P-40/20 mM phenylmethylsulfonyl fluoride) on a parafilm surface. After 6-9 min of extraction, the samples are fixed in 90% methanol/20 mM EGTA at −20° C. for a minimum of 10 minutes and are processed for immunofluorescence.

In Vitro Assembly of Paternal and Maternal Asters.

Centrosome-free lysates are prepared from Spisula oocytes 10, 20, and 40 minutes after KCl activation, are snap-frozen in liquid nitrogen, and stored at −80° C. Sperm heads with associated centrosomes are isolated and frozen in liquid nitrogen, and stored at −80° C. Frozen sperm heads are thawed and diluted in aster buffer (20 mM Pipes, pH 7.2/100 mM NaCl/5 mM MgSO₄) to a concentration of 4×10⁶/ml, which is determined by using a hemacytometer. Aliquots (10 μl) of lysates are thawed and mixed with 1 μl of the diluted sperm head solution (final concentration: 4×10⁵/ml), and the mixtures are incubated at room temperature for 15 min. Samples are then diluted with 1.5 ml of cold reassembly buffer (100 mM Pipes/1 mM EGTA/5 mM MgSO₄, pH 6.9) and placed on ice for 15 min to depolymerize microtubules that might have formed in the lysates, and then centrifuged for 5 min at 4° C. (6,000×g) with a Beckman JS13.1 swinging bucket rotor to pellet. The supernatant is aspirated carefully, leaving 20-30 μl of buffer overlaying the sperm head pellet. The pellet is resuspended, and samples are diluted in equal volume of ice-cold microtubule reassembly buffer containing 1 mg/ml sea urchin microtubule protein and GTP and are incubated at room temperature for 15 min to allow aster formation. The samples are fixed and sperm-heads and associated asters are immobilized onto coverslips and processed for immunofluorescence. For comparison, and as a control, the ability of isolated maternal Spisula centrosomes to assemble asters in 10, 20, and 40 min centrosome-free lysates is tested. Maternal centrosomes are isolated as described by Vogel and Palazzo (1997) and incubated in respective lysates at a final concentration of 4×10⁵/ml for 15 min at room temperature, are placed on ice for 15 min to depolymerize microtubules, are immobilized onto glass coverslips, are incubated with 0.5 mg/ml of sea urchin microtubule protein, and are processed for immunofluorescence.

In one embodiment of the invention, the screening method comprises a sperm aster assay wherein the sperm are treated with drug prior to mixing with lysate. Alternatively, drug can be added to the lysate before mixing with sperm. Failure to form asters would indicate the drug has an effect. This assay is particulary useful in the invention for screening potential therapeutic agents or drugs that may or are known to target the paternal centrosome and/or the maternal centrosome specifically.

Functional Reconstitution Assays

It will be appreciated by those of skill in the art that whole cell, gamete, or embryo assays such as the whole egg assay or the sperm aster assay need not always be performed prior to or in combination with other assays and that certain preparations or extracts of whole cell systems can be used to screen for potential therapeutic agents.

In one embodiment of the invention are functional reconstitution assays. These assays comprise the isolated centrosome assay and the centrosome remnant recovery assay, the mammalian hybrid assay and the tubulin polymerization assay, each of which are described below.

Centrosome Assembly Assays

Centrosome assembly and function is crucial for the coordinated polymerization of microtubules that leads to spindle assembly and chromosome segregation during mitosis. Thus, compounds that inhibit centrosome assembly and/or function may be useful anti-mitotic and anti-cancer agents. The isolated centrosome assay is used to (1) induce the synchronous assembly of centrosomes in hundreds of grams of live Spisula oocytes or large quantities of oocyte lysates and is performed according to the methods disclosed by Palazzo, R. E., Vaisberg, E., Cole, R. W. and Rieder, C. L. (1992) Science. 256: 219-221, (2) isolate large quantities of centrosomes for analysis under defined conditions and is performed according to the method described in Palazzo, R. E. and Vogel, J. M. (1999) In Mitosis and Meiosis: Methods in Cell Biology, vol. 61. C. L. Rieder, editor. Academic Press, Inc. 35-56, (3) disassemble and reconstitute functional centrosomes in vitro which are pefored according to Schnackenberg, B. J., Hull, D. R., Balczon, R. D. and Palazzo, R. E. (2000) J. Cell Sci. 113: 943-953, and (4) sperm centrosome assembly assays according to Wu, X. Y. and R. E. Palazzo (1999). Proc. Natl. Acad. Sci., 96: 1397-1402. These methods are incorporated by reference herein in their entirety.

In one embodiment of the invention, screening may be performed whereby isolated centrosomes or disassembled centrosome substructures are attached to glass, plastic, or any type of surface in test plates or coverslips and incubated in either centrosome-free cell extracts or defined tubulin media such that they function, or reassemble and recover function in the process of identifying potential therapeutic agents or drugs. In such methods, the function is easily determined as the formation of astral microtubule arrays observed with low power conventional light and fluorescent microscope objectives, or through visual or spectral chemiluminescence assays that determine tubulin content, is an indicator of astral microtubule content. Thus, inhibition of centrosome-dependent microtubule formation (aster formation) can be the basis of screens to identify compounds that inhibit centrosome assembly and function.

In this assay, centrosomes in assay plates are treated with tubulin media or cell extracts that may contain various compounds or varying concentrations of said compounds to be tested and the results can be observed by microscopy.

Centrosome Remnant Recovery Assay

The centrosome remnant assay used herein is performed according to the teachings of Schnackenberg, B. J., Hull, D. R., Balczon, R. D. and Palazzo, R. E. (2000) J. Cell Sci. 113: 943-953. It has been shown that the treatment of centrosomes with chaotropic agents such as KI (potassium iodide) and NaI (sodium iodide) yield a centrosome remnant or centromatrix that has lost the ability to nucleate microtubules. Thus microtubule nucleation potential may be regenerated in vitro by the addition of cell lysate from many animal cells. The result is the formation of asters with microtubules projecting radially from the centrosome. Methods of screening comprising a centrosome remnant recovery assay component are therefore contemplated by the present invention. In such screening methods, identification of drugs that interfere with the reconstitution of the centrosome would prevent the formation of asters and further define the target of the potential therapeutic agent or drug.

Hybrid Mammalian Assay

The hybrid mammalian assay used herein as a component of screening methods and drug discovery is performed according to the teachings of Schnackenberg, B. J., Hull, D. R., Balczon, R. D. and Palazzo, R. E. (2000) J. Cell Sci. 113: 943-953. It has been shown that certain aspects of spindle assembly, particularly those relating to centrosome assembly and chromosome capture by microtubules, can be reconstituted in hybrid systems using centrosomes isolated from Spisula oocytes and molecules and chromosomes isolated from mammalian transformed cells. Using these hybrid assays in combination with the whole gamete or cell-free functional reconstitution assays disclosed herein allow for optimization and selection of particular candidate therapeutic agents which may be useful as, or in the identification of, antimitotic agents that specifically target mammalian proteins required for spindle assembly.

Tubulin Polymerization Assay

The tubulin polymerization assay used herein is performed according to the teachings of Vogel, J. M., Steams, T., Rieder, C. L. and Palazzo, R. E. (1997) J. Cell Biol. 137: 193-202 and Suprenant K A, Marsh J C. (1987) J Cell Sci. 87:71-84, each of which is incorported herein by reference in its entirety.

Any assay described herein may be adapted for rapid throughput screening and/or high throughput screening and these may be further automated. Preferably the screening methods of the invention comprise multiwell plates, the advantage of this being that highthroughput screening can be achieved using industry standard components which can be processed using commercially available automated equipment and robotics. Any other types of high throughput screens, chips, glass or plastic slides may also be used. The invention will be further described with reference to the following examples; however, it is to be understood that the invention is not limited to such examples.

### EXAMPLES

The compound, HMN-176 ((E)-4-{[2-N-[4-methoxybenzenesulfonyl]amino]stillbazole}1-oxide) is an active metabolite of HMN-214 ((E)-4-{2-[2-(N-acetyl-N-[4-methoxybenzenesulfonyl]amino]stillbazole]}1-oxide), which has a potent antitumor activity on mouse xenograft models. While it has been reported that HMN-176 targets a transcription factor, the molecular mechanism of action of HMN-176 remains unknown (Tanaka et al., Cancer Res. 2003, 63; 6942-7). Investigations using the marine invertebrate gamete system of the present invention were undertaken to identify the target stage of development or site of action of HMN-176.

### Example 1

Whole Egg Germinal Vesicle Breakdown (GVBD) and Spindle Assembly Assays Showing Effect of Drug.

Using the whole egg assay described above, Spisula oocytes were treated with 0.25 μM HMN-176 or 0.001% DMSO and activated to undergo germinal vesicle (nucleus) breakdown (GVBD), and aster and spindle formation.

By 9-10 minutes, GBVD occurs and by 15 minutes spindles are visible in control cells. However, at the same 9 minute time point, cells treated with HMN-176 are undergoing GVBD but no spindles are detected by the 15 minute time point and for significant time after. By 26 minutes, spindles and asters are still visible in control cells but are not found in the HMN-176 cells. These data demonstrate that HMN-176, while not affecting GVBD (thereby eliminating as potential targets of HMN-176 several cell cycle dependent kinases), does inhibit spindle assembly in oocytes.

The effects seen here are similar to those of colchicine. Colchicine is a drug that has been reported to inhibit mitosis by significantly affecting tubulin polymerization and has been shown to be cytotoxic against several human tumor cell lines. Analysis of the effect of colchicine in the marine invertebrate gamete system shows that the drug does not affect GVBD but inhibits spindle assembly. The system as described distinguishes between a drug like colchicine which targets tubulin polymerization, and a drug like HMN-176 which has only mild effect on tubulin polymerization but also blocks spindle assembly through an alternative mechanism, a result of binding to an alternative target molecule that is not tubulin.

### Example 2

Effect of Drug on Aster Formation.

Using the lysate aster formation assay described above, Spisula lysates were warmed to room temperature with lysates containing varying amounts of HMN-176 (0.025 uM, 0.25 uM or 2.5 uM) or DMSO (0.0001%, 0.001%, or 0.01%) as control.

HMN-176 blocked aster formation in the lysate aster formation assay in a dose dependent manner, while DMSO (control) treated samples showed normal aster formation. To confirm the status of the centrosomes, samples were fixed and processed for immunofluorescence with antibodies to alpha-tubulin (green) and gamma-tubulin (red). The effect of double staining with green and red yields a yellow appearance to centrosomes in asters. In these studies, the centrosomes remained intact in all samples. These data indicate that HMN-176 inhibits centrosome-directed aster formation in extracts.

### Example 3

Effect of Drug on Aster Formation in Isolated Centrosome Assay

Using the isolated centrosome assay described above, HMN-176 was shown to block aster formation in defined media.

Isolated centrosomes reconstituted with 0.75 mg/mL sea urchin tubulin in defined media were treated with 0.25 uM HMN-176 and observed over a time course of 25 minutes with immunofluorescent observations being made at 5 minute intervals. Samples were stained with antibodies to alpha-tubulin (green) and gamma-tubulin (red). The time course revealed that isolated centrosomes reconstituted with cycled sea urchin tubulin in defined media formed asters in the DMSO treated samples. However, no asters were formed in the HMN-176 treated samples. In all HMN-176 treated samples microtubule polymerization was observed in the background (green) but no microtubules were associated with the centrosomes revealed by gamma-tubulin staining (red). In the control samples, microtubules were clearly associated with centrosomes forming normal asters. Centrosomes remained intact in all samples.

### Example 4

Effect of Drug on Tubulin Polymerization.

The effect of HMN-176 on sea urchin tubulin (0.75 mg/mL of S. purpuratus tubulin at 26° C.) polymerization in defined media was examined according to the methods described above. Sea urchin tubulin was isolated from Strongylocentrotus purpuratus oocytes by three cycles of microtubule polymerization and depolymerization. After the third cycle, microtubules were depolymerized, centrifuged to clarify, and the resulting supernatant adjusted to a concentration of 0.75 mg/ml protein. Samples were aliquoted and stored at −80° C.

The polymerization assay was used to test the effects of varying concentrations of HMN-176 (0.025 uM, 0.25 uM and 2.5 uM) along with varying amounts of DMSO for control (0.0001%, 0.001% and 0.1%), respectively. Polymerization of untreated tubulin was also measured. In all cases, HMN-176 had only minor effect on the polymerization of tubulin. The kinetics suggest that HMN-176 may slightly lower the rate of microtubule polymerization, but does not affect the ultimate plateau suggesting that by 25 minutes, tubulin polymerization in all samples is comparable based on the average turbidity at the end of the experiments. HMN-176, therefore, does not have a significant effect on tubulin polymerization.

Tubulin purity was confirmed by SDS-PAGE of both cytosol and lysates fractions of clam and sea urchin. Briefly, 4 μg of protein was loaded per lane and 5× lanes showed very dark tubulin bands, indicating high alpha/beta enrichment. No microtubule associated proteins were detected.

Sea urchin and clam tubulin turbidity assays were performed at 30° C. and 0.7 mg/mL of tubulin. Doses of 0.025 uM, 0.25 uM and 2.5 uM of HMN-176 were compared to DMSO treatment, all of which were compared to untreated control. Very little to no effect was seen in either the sea urchin or claim tubulin turbidity.

### Example 5

Effect of Drug on Mammalian Spindle Phenotypes.

To determine the effect of HMN-176 on mammalian cell lines, the transformed human cell line, CFPAC, was used. CFPAC cells were derived from a ductal adenocarcinoma (liver metastasis) from a patient with cystic fibrosis (ATCC #CRL-1918). CFPAC cellswere treated for 2 hours with 0.5 uM DMSO or 0.5 uM HMN-176 (n=79). Cells were studied for progression through mitosis and cell cleavage using video microscopy, or prepared for immunofluorescence analysis with antibodies to visualize beta tubulin or treated with Hoechst 33342 (Sigma Aldrich, St. Louis, Mo. to visualize DNA and chromosomes.

The time that CFPAC cells were in mitosis was found to be 147+/−55 minutes for HMN-176 treated cells as compared to 45+/−5 minutes for untreated cells. Observation of the spindle types revealed that generally, HMN-176 treated cells contained multipolar spindles with disorganized chromosomes. The data are summarized in Table 1 and suggest that HMN-176 does not affect the timing of some cell cycle events, but disrupts passage through mitosis by a mechanism that is independent of disruption of tubulin polymerization, possibly a disruption of centrosome function via disruption of centrosome-dependent microtubule nucleation and/or aster assembly.

TABLE 1
Spindle type upon treatment with drug

| Treatment | Spindle Type |
|---|---|
| 0.5 uM HMN-176 treatment for 2 hours | Monopolar 0 (0%) |
| (n = 79) | Bipolar 13 (16%) |
| | Multipolar 66 (84%) |
| 0.5 uM DMSO treatment for 2 hours | Monopolar 1 (2%) |
| | Bipolar 44 (88%) |
| | Multipolar 5 (10%) |

Taken together, the data from the foregoing examples on HMN-176 indicate that the drug is targeting the centrosome directly and most likely impacting centrosome-dependent microtubule nucleation. The data herein suggest that HMN-176 is either inhibiting the interaction of alpha-beta tubulin dimers with gamma tubulin or with gamma tubulin complexes (so called gamma TURCs or gamma TUSCs) or is transiently disrupting gamma tubulin ring complex structures reversibly thereby identifying the targeted stage of HMN-176 as involving tubulin polymerization. The effects seen are reversible in the assays of the present invention indicating that the drug is not causing permanent damage to any centrosome related structure. This makes the drug and similar agents attractive as anti-centrosome therapeutics.

The foregoing examples demonstrate that the Spisula marine invertebrate gamete system is an excellent model system for the discovery and screening of potential therapeutic agents as the information obtained in these assays can by used to obtain information on, and define the target of, known as well as unknown drugs. The results of these assays taken in combination also further the process of drug discovery of compounds with known and unknown targets. Consequently, not only can one identify new drugs targeting the centrosome using the embodiments of the present invention, one can also clarify the target of a compound with previously identified therapeutic activity. Finally, the ease and efficiency in terms of cost and time of implementing the MIGS, coupled with the visual readout make the system superior to high throughput assays or systems that require several analytical steps to reach a numeric endpoint.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. While this invention has been particularly shown and described with references to preferred embodiments thereof, it will be understood by those skilled in the art that various changes in form and details may be made therein without departing from the scope of the invention encompassed by the appended claims.

### REFERENCES

Palazzo, R. E., Vaisberg, E. A., Weiss, D. G., Kuznetsov, S. A. and Steffen, W. (1999). Dynein is required for spindle assembly in cytoplasmic extracts of Spisula solidissima oocytes. J. Cell Sci. 112: 1291-1302.
Palazzo, R. E., Vaisberg, E., Cole, R. W. and Rieder, C. L. (1992). Centriole duplication in lysates of Spisula solidissima oocytes. Science. 256: 219-221.
Vogel, J. M., Stearns, T., Rieder, C. L. and Palazzo, R. E. (1997). Centrosomes isolated from Spisula solidissima oocytes contain rings and an unusual stoichiometric ratio of alpha/beta-tubulin. J. Cell Biol. 137: 193-202.
Schnackenberg, B. J., Khodjakov, A., Rieder, C. L. and Palazzo, R. E. (1998). The disassembly and reassembly of functional centrosomes in vitro. Proc. Natl. Acad Sci. USA. 95: 9295-9300.
Wu, X. and Palazzo, R. E. (1999). Differential regulation of maternal vs paternal centrosomes. Proc. Natl. Acad. Sci. USA. 96: 1397-1402.
Schnackenberg, B. J. and Palazzo, R. E. (1999). Identification and function of the centrosome centromatrix. Biology of the Cell. 91: 429-438.
Dessev, G., Palazzo, R. E., Rebhun, L. I. and Goldman, R. (1989). Disassembly of the nuclear envelope of Spisula oocytes in a cell free system. Dev. Biol. 131: 496-504.
Palazzo, R. E. and Vogel, J. M. (1999). Isolation of centrosomes from Spisula solidissima oocytes. In Mitosis and Meiosis: Methods in Cell Biology, vol. 61. C. L. Rieder, editor. Academic Press, Inc. 35-56.
Schnackenberg, B. J., Hull, D. R., Balczon, R. D. and Palazzo, R. E. (2000). Reconstitution of microtubule nucleation potential in centrosomes isolated from Spisula solidissima oocytes. J. Cell Sci. 113: 943-953.

What is claimed is:

1. A method for screening potential therapeutic agents comprising:
a) isolating gametes or preparations thereof from a marine invertebrate;
b) contacting the gametes or preparations thereof of step (a) with one or more potential therapeutic agents;
c) quantifying the effect of the potential therapeutic agent on one or more phenotypic responses selected from the group consisting of aster formation, microtubule nucleation, nuclear envelope (germinal vesicle breakdown, GVBD), meiotic or mitotic spindle assembly, and embryonic cleavage; and
d) correlating a reduced phenotypic response of (c) in comparison to a non-contacted control with a therapeutic agent useful for treating a disease or condition.

2. The method of claim 1 wherein the disease or condition is selected from the group consisting of cancer, neuronal diseases or disorders, heart disease and reproductive conditions.

3. The method of claim 1 wherein the marine invertebrate is a clam, sea urchin, starfish or mussels.

4. The method of claim 3 wherein the marine invertebrate is the clam species Spisula solidissima or Mulinia lateralis.

5. The method of claim 1 wherein the gametes are oocytes.

6. The method of claim 1 wherein the gamete preparation comprises functional centrosomes.

7. The method of claim 6 wherein the functional centrosomes are purified.

8. The method of claim 1 wherein the gamete preparation comprises a cytoplasmic extract or lysate.

9. The method of claim 1 wherein quantification of (c) is effected visually by microscopy.

10. The method of claim 9 wherein the microscopy is polarized light microscopy.

11. The method of claim 1 wherein the potential therapeutic agent is selected from the group consisting of small organic compounds, small inorganic compounds, peptides, polypeptides, peptidomimetics, oligonucleotides, polynucelotides, aptamers and antibodies.

12. The method of claim 1 wherein the potential therapeutic agents are metabolites of known drugs.

13. A method of identifying compounds that disrupt centrosome-mediated cellular processes as potential therapeutic agents comprising:
a) isolating gametes and embryos or preparations thereof from a marine invertebrate;
b) contacting the gametes or preparations thereof of step (a) with one or more potential therapeutic agents;
c) quantifying the effect of the potential therapeutic agent on one or more centrosome-mediated cellular process; and
d) correlating an inhibition of the centrosome-mediated cellular process of (c) in comparison to a non-contacted control with a therapeutic agent that disrupt centrosome-mediated cellular processes and is useful for treating a disease or condition.

14. The method of claim 13 wherein the disease or condition is selected from the group consisting of cancer, neuronal diseases or disorders, heart disease and reproductive conditions.

15. The method of claim 13 wherein the centrosome-mediated cellular response is selected from the group consisting of aster formation, microtubule nucleation, nuclear envelope (germinal vesicle breakdown, GVBD), spindle assembly, and embryonic cleavage.

16. The method of claim 13 wherein the marine invertebrate is a clam.

17. The method of claim 16 wherein the marine invertebrate is the clam species Spisula solidissima or Mulinia lateralis.

18. The method of claim 13 wherein the gametes are oocytes.

19. The method of claim 13 wherein the gamete preparation comprises functional centrosomes.

20. The method of claim 19 wherein the functional centrosomes are purified.

21. The method of claim 13 wherein the gamete preparation comprises a cytoplasmic extract.

22. The method of claim 13 wherein quantification of (c) is effected visually via microscopy.

23. The method of claim 22 wherein the microscopy is polarized light.

24. The method of claim 13 wherein the potential therapeutic agent is selected from the group consisting of small organic compounds, small inorganic compounds, peptides, polypeptides, peptidomimetics, oligonucleotides, polynucelotides, aptamers and antibodies.

25. The method of claim 13 wherein the potential therapeutic agents are metabolites of known drugs.

26. The method of claim 12 wherein the metabolite is HMN-176.

27. The method of claim 25 wherein the metabolite is HMN-176.

28. A high throughput screening method comprising:
screening a library of potential therapeutic agents using a marine invertebrate whole gamete assay in combination with one or more cell-free functional reconstitution assays.

29. The high throughput screening method of claim 28 wherein the gametes are marine invertebrate Spisula solidissima oocytes or sperm.

30. The method of claim 28 wherein the cell-free functional reconstitution assay is selected from the group consisting of the oocyte lysate assay, the isolated centrosome assay, the centrosome remnant recovery assay, the activation of sperm centrosome assay, the mammalian hybrid assay and the tubulin polymerization assays.

31. The method of claim 28 wherein the whole gamete or embryo assay is performed in parallel with the cell-free functional reconstitution assay.
